(19) United States
(12) Patent Application Publication
(10) Pub. No.: US 2011/0077456 A1
(43) Pub. Date: Mar. 31, 2011

(54) PROLAPSE REPAIR DEVICE AND METHODS OF USE

(76) Inventor: Bruce Drummond, Madison, WI (US)

(21) Appl. No.: 12/895,330

(22) Filed: Sep. 30, 2010

Related U.S. Application Data
(60) Provisional application No. 61/247,257, filed on Sep. 30, 2009.

(51) Int. Cl. A61F 2/02

(52) U.S. Cl. 600/30

(57) ABSTRACT

An implantable prolapse repair device comprising a central graft material suitable for placement inside a human and first, second, and third mesh strips, suitable for placement inside a human body. The first mesh strip is operatively coupled to the central graft material by weaving the first mesh strip through the at least two slits to form a supportive weave. The second and third mesh strips have free ends, including a needle attached thereto and connector ends. The connector ends are also coupled to the central graft material. The central graft material may comprise human dermis or decellularized animal tissue.

### CROSS-REFERENCE TO RELATED APPLICATIONS

This application claims the benefit of U.S. Provisional Application No. 61/247,257, filed Sep. 30, 2009, the content of which is incorporated herein by reference in its entirety.

### FIELD OF THE INVENTION

An anterior vaginal wall prolapse occurs when the vaginal wall fails to hold the bladder in place. Anterior vaginal wall prolapse may present as one of two defects. Paravaginal defect is caused by weakness in the lateral supports (pubourethral ligaments and attachment of the bladder to the endopelvic fascia), and central defect is caused by weakness in the central supports. A posterior vaginal wall prolapse occurs when the rectum descends into the vagina. The severity of the prolapse is typically rated from stage I to stage IV, with stage IV being the most severe. Other prolapse conditions are known, including the presence of small intestine in a hernia sac between the rectum and vagina, called an enterocele.

Several factors have been implicated as contributing genital prolapse in women. Genetic differences result in individual women having different inherent strength of the relevant connective tissue. Environmental and behavioral differences also play a role, however. For example, loss of connective tissue strength might be associated with damage at childbirth, deterioration with age, poor collagen repair mechanisms, and poor nutrition. Loss of muscle strength might be associated with neuromuscular damage during childbirth, neural damage from chronic straining, and metabolic diseases that affect muscle function. Other factors involved in prolapse include increased loads on the supportive system, as seen in prolonged lifting or chronic coughing from chronic pulmonary disease, or some disturbance in the balance of the structural support of the genital organs. Obesity, constipation, and a history of hysterectomy have also been implicated as possible factors.

There are generally two different types of tissue that make up the supportive structure of the vagina and uterus. First, there are fibrous connective tissues that attach these organs to the pelvic walls (cardinal and uterosacral ligaments; pubocervical and rectovaginal fascia). Second, the levator ani muscles close the pelvic floor so the organs can rest on the muscular shelf thereby provided. It is when damage to the muscles opens the pelvic floor or during the trauma of childbirth that the fascia and ligaments are strained. Breaks in the fascia allow the wall of the vagina or cervix to prolapse downward.

Treatment for vaginal prolapse varies based on the symptoms of the prolapse as well as the health and age of the patient. If symptoms are more severe, treatment is commonly by either surgery or pessary. Surgical options might include hysterectomy, however newer sling methods have reduced the incidence of hysterectomy associated with prolapse repairs. Such procedures may include abdominal or vaginal access routes. Sacralcolpopexy or sacrospinous fixation may be used.

Unfortunately, many women who undergo surgery to repair prolapsed organs experience re-prolapse, pain, or infection. The high failure rate for prolapse repair has motivated the development of a number of implants to augment the known prolapse repair methods. Studies indicate that the implants have reduced the recurrence of prolapse as well as the morbidity associated with prolapse repair. Among the more successful implants, synthetic mesh systems for prolapse repair such as PINNACLE (Boston Scientific, Natick, Mass.) and ELEVATE (American Medical Systems, Minnetonka, Minn.) provide a means to support prolapsed organs by securing a mesh sling to various anatomical points within the pelvis. The mesh repair systems have proven especially beneficial for women who have weak tissues that otherwise would not support conventional prolapse repair. While mesh systems provide many benefits, long term results suggest an unacceptable rate of erosion of tissue, which can cause additional complications and require subsequent surgical procedures and hospitalization. Erosion rates between 10-20% have been reported (Watson, J. Am. Coll. Surg., vol. 183, p. 257 (1996)). In a recent study containing 43 cases the Pinnacle Pelvic Floor Repair Kit (Boston Scientific, Natick, Mass.) was used to correct anterior and apical pelvic floor defects. The study found a mesh erosion rate of about 27.9% and a surgical re-operation rate of about 14%. (Female Pelvic Medicine & Reconstructive Surgery: March/April 2010—Volume 16—Issue 2—S19).

To avoid erosion of tissue by the mesh, medical device manufacturers have introduced dermis-based systems comprising human (allograft) or animal (xenograft) dermis, such as REPLIFORM (cadaver dermis) and XENFORM (bovine dermis), both from Boston Scientific. These dermis based-systems are typically affixed to one or more ligaments to provide support to the pelvic organs. While the dermis-based systems have lower rates of erosion, the dermis systems are not as simple to secure as the mesh-based systems, often requiring a larger dissection plane. Additionally, the dermis-based systems do not offer consistent support for ingrown tissues as the dermis material, like human tissue, stretches and tears.

Other hybrid systems have been developed to allow the incorporation of materials other than mesh. Several examples are shown in U.S. Published Patent Application No. 2008/0081945, published Apr. 3, 2008, and incorporated by reference herein. These systems offer easier placement than dermis, and because the sling may be made from non-mesh material, erosion complications may be minimized. For example, FIG. 1 of U.S. Published Patent Application No. 2008/0081945 shows a sling assembly with a trapezoidal sling and four support arms to aid securing the sling.

### SUMMARY

The invention provides, among other things, an implantable prolapse repair device comprising a central graft material suitable for placement inside a human and first, second, and third mesh strips, suitable for placement inside a human body. The central graft material has at least two slits, a first connection point and a second connection point. The first mesh strip has a first free end, including a first needle attached thereto, and a second free end, including a second needle attached thereto. The first mesh strip is operatively coupled to the central graft material by weaving the first mesh strip through the at least two slits to form a supportive weave. The second mesh strip has a free end, including a needle attached thereto, and a connector end. The connector end of the second mesh strip is coupled to the first connection point of the central graft material. The third mesh strip has a free end, including a needle attached thereto, and a connector end. The connector end of the third mesh strip is coupled to the second connection point of the central graft material. The central graft material may comprise dermis or decellularized animal tissue. Each of the mesh strips may comprise at least one of polypropylene, polyethylene, polylactic acid (PLA), polyglycolic acid (PGA), poly-L-lactic acid (PLLA), polypeptides, and combinations thereof.

The invention additionally provides, among other things, a method for the repair of a prolapsed organ within a pelvis of a female using an implantable prolapse repair device of the invention. The method comprises dissecting anatomical structures beyond a vaginal wall through an incision in the vaginal wall to create a dissection plane, wherein the dissection plane has a first end and a second end of the anatomical structures, inserting an implantable prolapse repair device of the invention through the incision in the vaginal wall, coupling a cephalad end of the central graft material to a cervix or a vaginal cuff, coupling the first free end of the first mesh strip to a first sacrospinous ligament, coupling the second free end of the first mesh strip to a second sacrospinous ligament, coupling the free end of the second mesh strip to a first arcus tendineous, coupling the free end of the third mesh strip to a second arcus tendineous and suturing the central graft material to the second end of the anatomical structures defined by the dissection plane. In many embodiments, the supportive weave is spaced apart from the vaginal wall by the central graft material. The method may additionally comprise performing a site-specific repair.

The invention additionally provides, among other things, a prolapse repair kit comprising a central graft material suitable for placement inside a human and first, second, and third mesh strips, suitable for placement inside a human body. The central graft material has two slits and first and second connection points. The first mesh strip has a first free end, including a first needle attached thereto, and a second free end, including a second needle attached thereto. The second mesh strip has a free end, including a needle attached thereto, and a connector end. The third mesh strip has a free end, including a needle attached thereto, and a connector end. The two slits in the central graft material allow passage of the first mesh strip through the central graft material to form a supportive weave such that the first and second free ends extend outwardly from the central graft material. The connector end of the second mesh strip is coupleable to the first connection point of the central graft material, and the connector end of the third mesh strip is coupleable to the second connection point of the central graft material. The central graft material may comprise human dermis or decellularized animal tissue. Each of the mesh strips may comprise at least one of polypropylene, polyethylene, polylactic acid (PLA), polyglycolic acid (PGA), poly-L-lactic acid (PLLA), polypeptides, and combinations thereof.

The invention additionally provides, among other things, a method of making a prolapse repair device, comprising passing a first mesh strip through a central graft material having two slits, thereby forming a supportive weave, the first mesh strip having a first free end, including a first needle attached thereto, and a second free end, including a second needle attached thereto, the first and second free ends extending outwardly from the central graft material, coupling a connector end of a second mesh strip to a first connection point of the central graft material such that the free end of the second mesh strip extends outwardly from the central graft material, and coupling a connector end of a third mesh strip to a second connection point of the central graft material such that the free end of the second mesh strip extends outwardly from the central graft material.

Other aspects of the invention will become apparent by consideration of the detailed description and accompanying drawings.

### BRIEF DESCRIPTION OF THE DRAWINGS

FIG. 1 illustrates a prolapse repair device in accordance with at least one embodiment of the present invention.

FIG. 2 shows an enlarged view of the prolapse repair device illustrated in FIG. 1.

FIG. 3 illustrates a kit used to assemble the prolapse repair kit shown in FIG. 2 in accordance with at least one embodiment of the present invention.

FIG. 4A illustrates placement of a first mesh strip of the prolapse repair device in a sacrospinous ligament.

FIG. 4B illustrates placement of a second mesh strip of the prolapse repair device in the arcus tendineous.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. The figures referred to herein are not necessarily to scale; emphasis instead is generally placed upon illustrating the principles of the illustrated embodiments.

### DETAILED DESCRIPTION

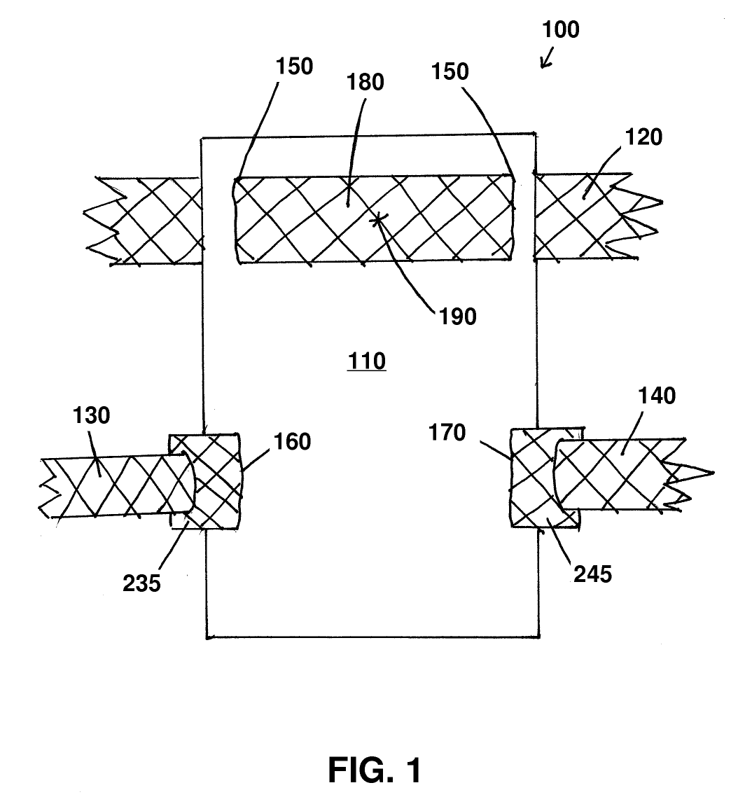

The invention discloses an improved prolapse repair devices that offers superior results when used to repair prolapsed organs. In one embodiment, shown in FIG. 1, an implantable prolapse repair device 100 comprises a central graft material 110, a first mesh strip 120, a second mesh strip 130, and a third mesh strip 140. The central graft material 110, first mesh strip 120, second mesh strip 130, and third mesh strip 140 are all suitable for placement inside a human body. The central graft material 110 has at least two slits 150, as well as a first connection point 160 and a second connection point 170. (First connection point 160 and second connection point 170 need not be on a particular side of central graft material 110, and are named “first” and “second” for convenience). In a further embodiment, the central graft material 110 may include more than two slits 150. By example, the graft material 110 can include 3, 4, 5, 6 or more slits. The first mesh strip 120 is operatively coupled to the central graft material 110 by weaving the first mesh strip 120 through the at least two slits 150 to form a supportive weave 180. (Supportive weave 180 need not be made from a woven material, however.) An anchor suture 190 can be used to couple the first mesh strip 120 and the central graft material 110 to restrict motion there between. Alternatively, the graft material is presented with out connection points 160, 170, and the device utilizes one or more mesh strips.

The central graft material 110 can be constructed of any materials acceptable for placement within humans that have suitable mechanical properties. In some embodiments, the central graft material 110 can comprise dermis or decellularized animal tissue. The decellularized animal tissue can comprise, but need not be limited to, bovine tissue, porcine tissue, ovine tissue, or equine tissue. The central graft material 110 may be an omnidirectional material, a material that has equivalent tensile strength from any direction, such as pericardium or dermis. Alternatively, the central graft material 110 may be an oriented material, having a single direction where the tensile strength of the material is the highest. Oriented materials may include rectus fascia and/or fascia lata. Mesh strips 120, 130, and 140 may be constructed of any materials acceptable for placement within humans that have suitable mechanical properties. While the materials need not be mesh, open materials such as mesh, weave, open fabrics, etc., allow for ingrowth of cellular material, thus stabilizing the implantable prolapse repair device 100. In some embodiments, the mesh strips 120, 130, and 140 may be constructed from polypropylene, polyethylene, polylactic acid (PLA), polyglycolic acid (PGA), poly-L-lactic acid (PLLA), polypeptides, combinations thereof, or other suitable materials.

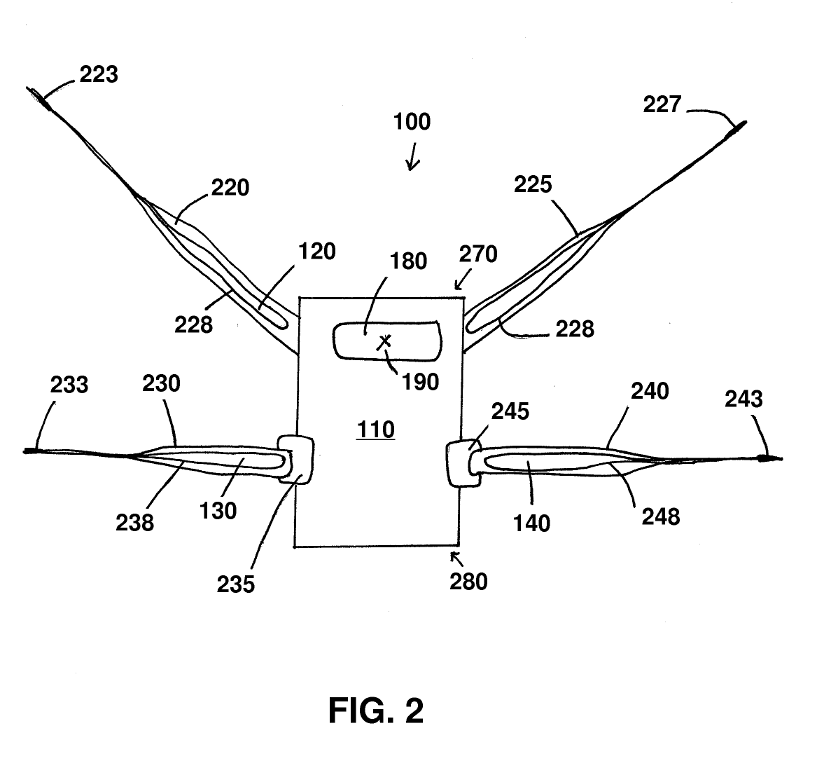

As shown in FIG. 2, the first mesh strip 120 has a first free end 220, including a first needle 223 attached thereto. The first mesh strip 120 also has second free end 225, including a second needle 227 attached thereto. The second mesh strip 130 has free end 230, including a needle 233 attached thereto, and a connector end 235. The connector end 235 of the second mesh strip 130 is coupled to the first connection point 160 of the central graft material 110. The third mesh strip 140 has a free end 240, including a needle 243 attached thereto, and a connector end 245. The connector end 245 of the third mesh strip 140 is coupled to the second connection point 170 of the central graft material 110. As shown in FIG. 2, free ends 220, 225, 230, and 240 may be arranged to extend outwardly from the central graft material 110. The first mesh strip 120 may additionally include a reinforcing wire 228, the second mesh strip 130 may additionally include a reinforcing wire 238, and the third mesh strip 140 may additionally include a reinforcing wire 248. The first mesh strip 120 may include a contiguous reinforcing wire, or multiple reinforcing wires to avoid pressure points along the central graft material 110. Needles 223, 227, 233, 243 may be constructed of stainless steel, titanium, or other suitable materials. Needles 223, 227, 233, 243 need not be true needle structures, and may, for example, be small sharpened or blunt tips, as necessary to interface with implantation devices, such as CAPIO (Boston Scientific).

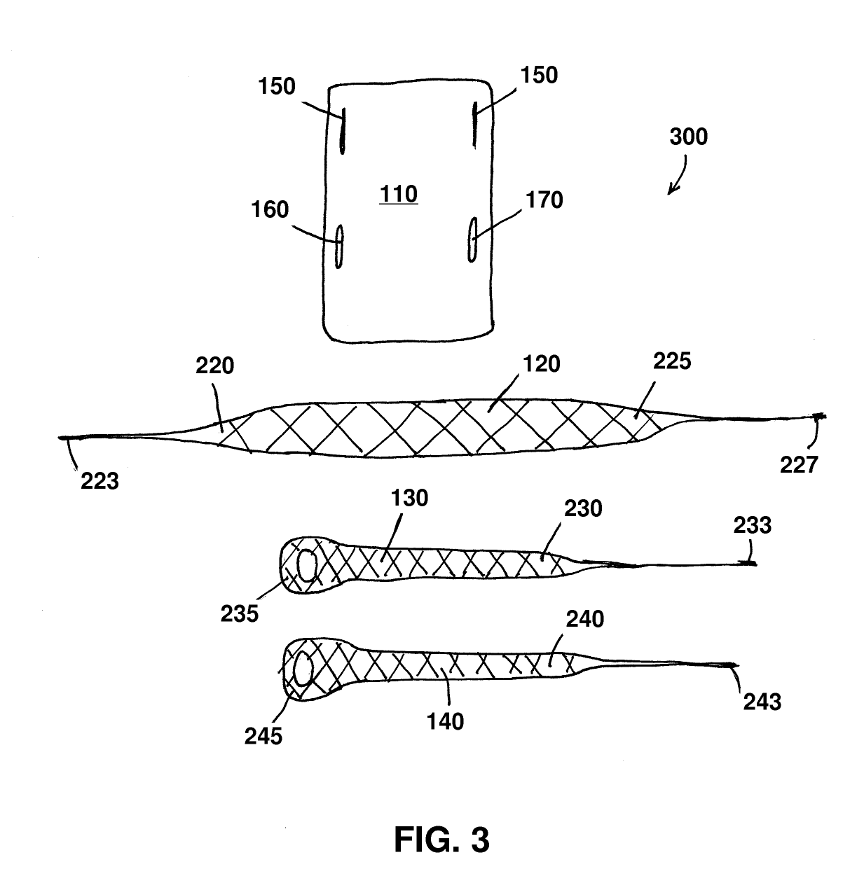

As shown in FIG. 3, the implantable prolapse device 100 may be sold as a prolapse repair kit 300, comprising the central graft material 110, the first mesh strip 120, the second mesh strip 130, and the third mesh strip 140, as described above. In alternative embodiments, prolapse repair kit 300 may contain a singular piece of mesh (not shown) from which mesh strips 120, 130, and 140 may be fabricated. For example, a singular piece of mesh with free ends with needles (or similar structures) attached thereto can be cut to create mesh strips 120, 130, and 140, and the newly-fabricated mesh strips incorporated into an implantable prolapse repair kit of the invention.

In some embodiments, elements of the prolapse repair kit 300 may be separately packaged because of separate storage needs, e.g., the central graft material 110 may need to stay hydrated in saline, and thus, is not packaged with mesh strips 120, 130, or 140. In other embodiments, pieces of the prolapse repair kit may be sold separately, with the surgeon being instructed how to assemble the pieces to create an implantable prolapse repair device according to the invention.

The implantable prolapse repair device 100 is assembled from the prolapse repair kit 300 by passing the first mesh strip 120 through the central graft material 110 using the at least two slits 150, thereby forming the supportive weave 180. In the illustrated embodiment, the anchor suture 190 is used to couple the first mesh strip 120 and the central graft material 110 to restrict motion there between, although in further embodiments an anchor suture may not be necessary. The first mesh strip 120 is arranged such that the first free end 220 and the second free end 225 extend outwardly from the central graft material 110, as is shown in FIG. 2. Next, the connector end 235 of the second mesh strip 130 is coupled to the first connection point 160 of the central graft material 110 such that the free end 230 of the second mesh strip 130 extends outwardly from the central graft material 110, as is shown in FIG. 2. Finally, the connector end 245 of the third mesh strip 140 is coupled to the second connection point 170 of the central graft material 110 such that the free end 240 of the third mesh strip 140 extends outwardly from the central graft material 110, as is shown in FIG. 2, thereby producing a complete implantable prolapse repair device 100.

In the illustrated embodiment, each of the connections points 160, 170 are slits formed in the central graft material 110. The second and third mesh strips 130, 140 are coupled to the central graft material 110 by looping mesh strips 130, 140 through the connection points 160, 170. Each of the connector ends 235, 245 are passed through the respective connection points 160, 170. Each of the free ends 230, 240 of mesh strips 130, 140 is passed through an opening formed in the respective connector end 235, 245 to couple mesh strips 130, 140 to the central graft material 110. In further embodiments, the mesh strips 130, 140 are coupled to the central graft material 110 by other known means.

An implantable prolapse repair device according to the invention may be used to repair prolapsed organs in the female pelvis using the following technique: The patient is placed in the dorsal lithomy position and is prepped and draped as standard. An incision is made in the anterior vaginal wall below the level of the vaginal muscularis to maintain a vascularized epithelium. A finger is inserted through the incision to create a dissection plane. At this point, one or more optional site-specific repairs such as a midline vaginal defect repair, paravaginal plication of the anterior wall, a posterior repair of the rectovaginal fascia, or a repair of the pubocervical fascia, can be performed. These repairs, typically done with absorbable suture, can be completed prior to implanting the prolapse repair device. Additionally, these repairs are often advisable when the repair device incorporates a central graft material containing dermis, decellularized animal tissue or other similar material.

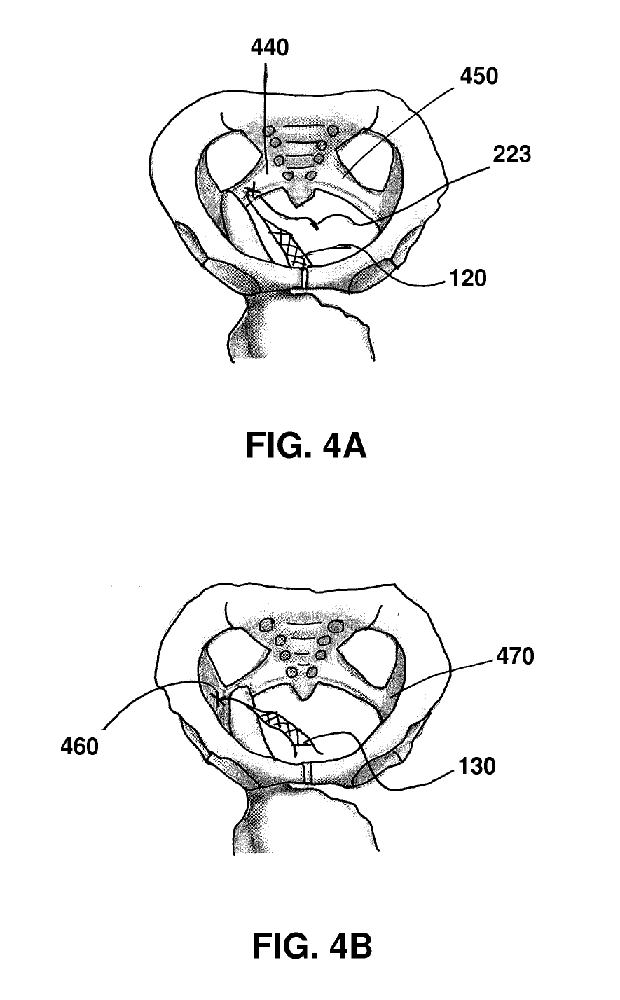

Next, an implantable prolapse repair device is inserted through the incision in the vaginal wall, and a cephalad end 270 of the central graft material 110 is affixed to the cervix or vaginal cuff. The central graft material is attached such that when the prolapse repair device is placed, the supportive weave will be separated from the vaginal wall by the central graft material. A CAPIO Device (Boston Scientific, Natick Mass.) is then loaded with the needle attached to first mesh strip free first end 223. A sacrospinous ligament 440 on the corresponding side of the patient is located by dissecting with a finger. The CAPIO device is then located adjacent to the finger, contacting the sacrospinous ligament 440, and activated, causing the needle attached to first mesh strip first free end 223 to pass through the sacrospinous ligament 440 as illustrated in FIG. 4A, resulting in the first mesh strip 120 being coupled to the sacrospinous ligament 440. In a similar fashion, the needle attached to first mesh strip second free end 227 is attached to a contralateral sacrospinous ligament 450. A suture can be placed through a central portion of the mesh and a central portion of the cervix or vaginal cuff in order to prevent and/or lessen the likelihood of slippage of the graft from the apex of the vagina.

Following attachment of the first strip 120 to the sacrospinous ligaments 440 and 450, the finger is used to locate an arcus tendineous 460 in order to secure the second mesh strip 130. The CAPIO device is again located adjacent to the finger, contacting the arcus tendineous 460, and activated, causing the needle attached to the second mesh strip free end 233 to pass through arcus tendineous 460, as illustrated in FIG. 4B, resulting in second mesh strip 130 being coupled to the arcus tendineous 460. In a similar fashion, the needle attached to the third mesh strip free end 243 is attached to a contralateral arcus tendineous 470. At this point, the strips 120, 130, 140, may be adjusted to achieve the desired support, and free ends 220, 225, 230, and 240 trimmed and removed from the patient. Prior to closing the incision, a caudal portion 280 of the central graft material 110 is sutured to the caudal portion of the anterior dissection. The vaginal incision is then closed with suture.

In a similar fashion, the implantable prolapse repair device 100 may be placed via a posterior vaginal incision. Additionally, an embodiment (not shown) of the repair device of FIG. 1-2 is presented absent the left and right Arcus mesh arms. Without these arms, complications from mesh placement may be reduced. Use of the device with or without the right and left arcus mesh arms can be selected based upon a surgeon's preference and/or based upon a particular patient's medical condition. The alternative embodiment can include a substantially smaller portion of the central graft material 110, and can be prefabricated or cut to the desired dimensions by the surgeon.

The central graft material 110 and mesh strips 120, 130, and 140 may include one or more agents for release into a patient's tissues. In one embodiment, the agent may be a tissue growth factor that, when applied to the patient's tissues in a pharmaceutically acceptable amount, promotes well-organized collagenous tissue growth, such as scar tissue growth, preferably, in large quantities. According to one feature, the agent may or may not block or delay the dissolvability of the biodegradable materials. Whether or not an agent blocks or delays such dissolvability may be controlled by selecting differing methods for loading the agent onto the sling. Exemplary tissue growth factors may include natural and/or recombinant proteins for stimulating a tissue response to enhance collagenous tissue growth. Exemplary growth factors that may be used include, but are not limited to, platelet-derived growth factor (PDGF), fibroblast growth factor (FGF), transforming growth factor-beta (TGF-beta), vascular endothelium growth factor (VEGF), Activin/TGF and sex steroid, bone marrow growth factor, growth hormone, Insulin-like growth factor 1, and combinations thereof. The agent may also include a hormone, including but not limited to estrogen, steroid hormones, and other hormones to promote growth of appropriate collagenous tissue such as scar tissue. The agent may also include stem cells or other suitable cells derived from the host patient. These cells may be fibroblast, myoblast, or other progenitor cells to mature into appropriate tissues.

In some embodiments, the agent may additionally comprise one or more therapeutic agents. The therapeutic agents may be, for example, anti-inflammatory agents, including steroidal and non-steroidal anti-inflammatory agents, analgesic agents, including narcotic and non-narcotic analgesics, local anesthetic agents, antispasmodic agents, growth factors, gene-based therapeutic agents, and combinations thereof. Exemplary steroidal anti-inflammatory therapeutic agents (glucocorticoids) include, but are not limited to, 21-acetoxyprefnenolone, aalclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumehtasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol priopionate, halometasone, halopredone acetate, hydrocortamate, hydrocortisone, loteprednol etabonate, mazipredone, medrysone, meprednisone, methyolprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, triamcinolone hexacetonide, and pharmaceutically acceptable salts thereof.

Exemplary non-steroidal anti-inflammatory therapeutic agents include, but are not limited to, aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid; carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as c-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; and pharmaceutically acceptable salts thereof.

Exemplary narcotic analgesic therapeutic agents include, but are not limited to, alfentanil, allylprodine, alphaprodine, anileridine, benzylmorphine, bezitramide, buprenorphine, butorphanol, clonitazene, codeine, codeine methyl bromide, codeine phosphate, codeine sulfate, desomorphine, dextromoramide, dezocine, diampromide, dihydrocodeine, dihydrocodeinone enol acetate, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone hydrochloride, metopon, morphine, myrophine, nalbuphine, narceine, nicomorphine, norlevorphanol, normethadone, normorphine, norpipanone, opium, oxycodone, oxymorphone, papavereturn, pentazocine, phenadoxone, phenazocine, pheoperidine, piminodine, piritramide, proheptazine, promedol, properidine, propiram, propoxyphene, rumifentanil, sufentanil, tilidine, and pharmaceutically acceptable salts thereof.

Exemplary non-narcotic analgesic agents that may be combined with the slings of the invention include, but are not limited to, aceclofenac, acetaminophen, acetaminosalol, acetanilide, acetylsalicylsalicylic acid, alclofenac, alminoprofen, aloxiprin, aluminum bis(acetylsalicylate), aminochlorthenoxazin, 2-amino-4-picoline, aminopropylon, aminopyrine, ammonium salicylate, amtolmetin guacil, antipyrine, antipyrine salicylate, antrafenine, apazone, aspirin, benorylate, benoxaprofen, benzpiperylon, benzydamine, bermoprofen, brofenac, p-bromoacetanilide, 5-bromosalicylic acid acetate, bucetin, bufexamac, bumadizon, butacetin, calcium acetylsalicylate, carbamazepine, carbiphene, carsalam, chloralantipyrine, chlorthenoxazin(e), choline salicylate, cinchophen, ciramadol, clometacin, cropropamide, crotethamide, dexoxadrol, difenamizole, diflunisal, dihydroxyaluminum acetylsalicylate, dipyrocetyl, dipyrone, emorfazone, enfenamic acid, epirizole, etersalate, ethenzamide, ethoxazene, etodolac, felbinac, fenoprofen, floctafenine, flufenamic acid, fluoresone, flupirtine, fluproquazone, flurbiprofen, fosfosal, gentisic acid, glafenine, ibufenac, imidazole salicylate, indomethacin, indoprofen, isofezolac, isoladol, isonixin, ketoprofen, ketorolac, p-lactophenetide, lefetamine, loxoprofen, lysine acetylsalicylate, magnesium acetylsalicylate, methotrimeprazine, metofoline, miroprofen, morazone, morpholine salicylate, naproxen, nefopam, nifenazone, 5′ nitro-2′ propoxyacetanilide, parsalmide, perisoxal, phenacetin, phenazopyridine hydrochloride, phenocoll, phenopyrazone, phenyl acetylsalicylate, phenyl salicylate, phenyramidol, pipebuzone, piperylone, prodilidine, propacetamol, propyphenazone, proxazole, quinine salicylate, ramifenazone, rimazolium metilsulfate, salacetamide, salicin, salicylamide, salicylamide o-acetic acid, salicylsulfuric acid, salsalte, salverine, simetride, sodium salicylate, sulfamipyrine, suprofen, talniflumate, tenoxicam, terofenamate, tetradrine, tinoridine, tolfenamic acid, tolpronine, tramadol, viminol, xenbucin, zomepirac, and pharmaceutically acceptable salts thereof.

Exemplary local anesthetic therapeutic agents include, but are not limited to, ambucaine, amolanone, amylocalne hydrochloride, benoxinate, benzocaine, betoxycaine, biphenamine, bupivacaine, butacaine, butaben, butanilicaine, butethamine, butoxycaine, carticaine, chloroprocaine hydrochloride, cocaethylene, cocaine, cyclomethycaine, dibucaine hydrochloride, dimethisoquin, dimethocaine, diperadon hydrochloride, dyclonine, ecgonidine, ecgonine, ethyl chloride, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocalne, procaine, propanocaine, proparacaine, propipocaine, propoxycaine hydrochloride, pseudococaine, pyrrocaine, ropavacaine, salicyl alcohol, tetracaine hydrochloride, tolycaine, trimecaine, zolamine, and pharmaceutically acceptable salts thereof.

Exemplary antispasmodic therapeutic agents include, but are not limited to, alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-ltrimethyl-3,3-diphenyl-propylamine, tropenzile, trospium chloride, xenytropium bromide, and pharmaceutically acceptable salts thereof.

The implantable prolapse repair device 100 is expected to have favorable long-term outcomes when implanted as described above. One improvement over the existing prolapse repair devices, such as described in U.S. Published Patent Application No. 2008/0081945, is the use of the supportive weave 180, which allows the implantable prolapse repair device 100 to provide mesh-like support to prolapsed organs (e.g., vagina, bladder, rectum, uterus) while not actually contacting the vaginal wall with mesh material. In particular, benefits are anticipated when the supportive weave is spaced apart from the vaginal wall by the central graft material. Such an arrangement provides improved support with reduced risk of erosion. Additionally, the prior attachment of needles to free ends 220, 225, 230, and 240 allows for fast and accurate implantation. Other benefits may include lower incidence of post-operative bleeding, infection, and re-operation.

### EXAMPLES

### Example 1

### Prolapse Repair Using Implantable Prolapse Repair Device

A 68-year-old woman presented with complaints of urinary discomfort and incontinence. A pelvic exam indicated anterior vaginal wall prolapse. After pre-op and prep, a longitudinal anterior vaginal incision was made and a dissection plane was established with sharp and blunt disection. Prior to implanting the prolapse repair device, a site specific repair was performed, repairing the pubocervical fascial defect using absorbable suture. Following the fascial repair, an implantable prolapse repair device similar to the device shown in FIG. 2 was placed beyond the dissection plane, and the cephalad end of the central graft material was attached to the midline of the vaginal cuff with suture. Following attachment of the cephalad end, the free ends of the first mesh strip were passed through the left and right sacrospinous ligaments, using the attached needles, by using a CAPIO Device (Boston Scientific, Natick, Mass.). The central area of the mesh was sutured to the midline of the vaginal cuff. Next, the free ends of the second and third mesh strips were passed through the left and right arcus tendineous using the corresponding attached needles and a CAPIO Device. The free ends were then trimmed and the remnant free ends removed. After securing the free ends, the caudal end of the central graft material was attached to the caudal end of the anterior dissection, and the incision closed. The patient stayed overnight at the hospital for observation and rested at home for approximately two weeks before resuming light activities. Eleven months post-operative, the patient reports no abnormal bleeding, discharge and no pain. No complications are known.

### Example 2

### Results of 19 Prolapse Repair Procedures

Using the techniques described in EXAMPLE 1, nineteen women of varying age, presenting Stage II or greater vaginal prolapse, underwent repair procedures without concurrent hysterectomy. The results are summarized in Table 1.

TABLE 1
Summary of prolapse repair procedures using the invention
after 2 years.

| Patient | Age | Repair | Months Post | Known Complications |
|---|---|---|---|---|
| 1 | 79 | Anterior | 23 | None |
| 2 | 68 | Anterior | 23 | Skin Dehiscence (1) |
| 3 | 76 | Anterior | 22 | Recurrent prolapse (2) |
| 4 | 48 | Anterior | 20 | Recurrent prolapse (3) |
| 5 | 56 | Anterior | 20 | None |
| 6 | 61 | Anterior | 20 | None |
| 7 | 41 | Anterior | 21 | Erosion(4) |
| 8 | 58 | Anterior | 18 | None |
| 9 | 81 | Anterior | 18 | None |
| 10 | 70 | Posterior | 14 | None |
| 11 | 63 | Anterior | 17 | None |
| 12 | 77 | Anterior | 15 | None |
| 13 | 57 | Anterior | 13 | None |
| 14 | 69 | Anterior | 11 | None |
| 15 | 66 | Anterior | 11 | None |
| 16 | 73 | Anterior | 11 | None |
| 17 | 69 | Anterior | 11 | None |
| 18 | 79 | Anterior | 11 | None |
| 19 | 76 | Posterior | 10 | None |
| 20 | 78 | Anterior | 9 | None |
| 21 | 77 | Anterior | 7 | Skin Dehiscence (5) |
| 22 | 75 | Anterior | 4 | None |
| 23 | 66 | Anterior | 3 | Skin Dehiscence (6) |

Overall, the results are encouraging. Patient follow-up indicates fewer mesh erosions than would be expected from a similar patient population undergoing mesh-only prolapse repair. Nonetheless, patients reported apical support similar to that provided by mesh-only systems. Patients 1, 21 and 23 each presented within 6 weeks post procedure with increased discharge and were noted to have 3 cm or less skin separation exposing the dermis without mesh erosions. These patients all resolved with oral or vaginal antibiotics. Patient 3, a 76 year old, presented at 14 months post procedure with a recurrent cystocele. The apex was well supported. A subsequent surgical repair was done to successfully repair the cystocele. Patient 4, a 48 year old, experienced a severe cough immediately after surgery and recurrence was noted at 6 weeks post procedure. The cervix was elongated. At 20 months, the patient had a total vaginal and uterosacral ligament fixation. Patient 7, a 41 year old, was found to have an apical mesh erosion at 6 weeks post procedure and this was corrected by removing the exposed mesh in the midline, at an office visit soon thereafter, the right arcus tendineus mesh attachment was removed in the operating room.

The singular mesh erosion noted in Table 1 was corrected by removing the exposed mesh during an office visit; the patient did not experience any recurrent anatomical failure thereafter. The recurrent anterior prolapse noted in Table 1 required re-operation and placement of new mesh over the anterior vaginal wall. The new mesh was attached to the cephalad mesh from the prior procedure, which remained affixed to the sacrospinous ligaments.

During the prodcedures listed in Table 1 there were no serious complications, such as large vessel injuries leading to hemorrhage, nerve injury, or serious infection. The patients experienced no significant post-operative chronic pain associated with graft placement. The patients represented in Table 1 have not reported any urinary retention as a result of the repair.

Thus, the invention provides, among other things, an implantable prolapse repair device, a method of using a prolapse repair device, a kit containing a prolapse repair device, and a method of making a prolapse repair device. Various features and advantages of the invention are set forth in the following claims.

What is claimed is:

1. An implantable prolapse repair device comprising:
a central graft material suitable for placement inside a human, the central graft material having at least two slits, a first connection point and a second connection point;
a first mesh strip suitable for placement inside a human and operatively coupled to the central graft material, the first mesh strip having a first free end including a first needle attached thereto, and a second free end including a second needle attached thereto, the first mesh strip woven through the at least two slits to form a supportive weave;
a second mesh strip suitable for placement inside a human, the second mesh strip having a free end including a needle attached thereto, and a connector end coupled to the first connection point; and
a third mesh strip suitable for placement inside a human, the third mesh strip having a free end including a needle attached thereto, and a connector end coupled to the second connection point.

2. The implantable prolapse repair device of claim 1, further comprising an anchor suture coupled to the first mesh strip and the central graft material to restrict motion there between.

3. The implantable prolapse repair device of claim 1, wherein the central graft material comprises human dermis or decellularized animal tissue.

4. The implantable prolapse repair device of claim 3, wherein the decellularized animal tissue is bovine tissue, porcine tissue, ovine tissue, or equine tissue.

5. The implantable prolapse repair device of claim 1, wherein each of the mesh strips suitable for placement inside a human comprises at least one of polypropylene, polyethylene, polylactic acid (PLA), polyglycolic acid (PGA), poly-L-lactic acid (PLLA), polypeptides, and combinations thereof.

6. The implantable prolapse repair device of claim 1, wherein the first mesh strip includes a reinforcing wire.

7. A method for the repair of a prolapsed organ within a pelvis of a female using the implantable prolapse repair device according to claim 1, the method comprising:
dissecting anatomical structures beyond a vaginal wall through an incision in the vaginal wall to create a dissection plane, the dissection plane defining a first end and a second end of the anatomical structures;
inserting the implantable prolapse repair device according to claim 1 through the incision in the vaginal wall;
coupling a cephalad end of the central graft material to a cervix or a vaginal cuff;
coupling the first free end of the first mesh strip to a first sacrospinous ligament;
coupling the second free end of the first mesh strip to a second sacrospinous ligament;
coupling the free end of the second mesh strip to a first arcus tendineous;
coupling the free end of the third mesh strip to a second arcus tendineous; and
suturing the central graft material to the second end of the anatomical structures defined by the dissection plane.

8. The method of claim 7, further comprising performing a site-specific repair through the incision in the vaginal wall.

9. The method of claim 8, wherein the site-specific repair comprises repairing the pubocervical fascia with suture.

10. The method of claim 7, wherein the supportive weave is spaced apart from the vaginal wall by the central graft material.

11. The method of claim 7, wherein the incision is formed in an anterior of the vaginal wall.

12. The method of claim 7, wherein the device, when implanted, contains the absorbable dermis between a synthetic mesh and vaginal mucosa.

13. A prolapse repair kit comprising:
a central graft material suitable for placement inside a human, the central graft material having two slits and first and second connection points;
a first mesh strip suitable for placement inside a human, the first mesh strip having a first free end, including a first needle attached thereto, and a second free end, including a second needle attached thereto;
a second mesh strip suitable for placement inside a human, the second mesh strip having a free end, including a needle attached thereto, and a connector end; and
a third mesh strip suitable for placement inside a human, the third mesh strip having a free end, including a needle attached thereto, and a connector end,
wherein the two slits in the central graft material allow passage of the first mesh strip through the central graft material to form a supportive weave such that the first and second free ends extend outwardly from the central graft material, wherein the connector end of the second mesh strip is coupleable to the first connection point of the central graft material, and wherein the connector end of the third mesh strip is coupleable to the second connection point of the central graft material.

14. The prolapse repair kit of claim 13, wherein the central graft material comprises human dermis or decellularized animal tissue.

15. The prolapse repair kit of claim 13, wherein the decellularized animal tissue is bovine tissue, porcine tissue, ovine tissue, or equine tissue.

16. The prolapse repair kit of claim 13, wherein each of the mesh strips suitable for placement inside a human comprises at least one of polypropylene, polyethylene, polylactic acid (PLA), polyglycolic acid (PGA), poly-L-lactic acid (PLLA), polypeptides, and combinations thereof.

17. The prolapse repair kit of claim 13, wherein the first mesh strip includes a reinforcing wire.

18. A method of making an implantable prolapse repair device, comprising:
passing a first mesh strip through a central graft material having two slits, thereby forming a supportive weave, the first mesh strip having a first free end, including a first needle attached thereto, and a second free end, including a second needle attached thereto, the first and second free ends extending outwardly from the central graft material;
coupling a connector end of a second mesh strip to a first connection point of the central graft material such that the free end of the second mesh strip extends outwardly from the central graft material; and
coupling a connector end of a third mesh strip to a second connection point of the central graft material such that the free end of the second mesh strip extends outwardly from the central graft material.

19. An implantable prolapse repair device comprising:
a central graft material comprising bovine tissue in combination with a mesh strip, the graft material having a plurality of slits through which the mesh strip is woven to form a supportive weave, the mesh strip being attached to the graft material, the mesh strip further comprising a first free end, including a first needle attached thereto, and a second free end, including a second needle attached thereto.

20. The device according to claim 19, further comprising a second mesh strip connected to the central graft material such that the free end of the second mesh strip extends outwardly from the central graft material and a third mesh strip connected to the central graft material such that the free end of the second mesh strip extends outwardly from the central graft material.
